(19) United States
(12) Patent Application Publication
(10) Pub. No.: US 2021/0146195 A1
(43) Pub. Date: May 20, 2021

(54) EXERCISE RECORDING DEVICES AND RELATED METHODS

(71) Applicant: Robert SZPICZYNSKI, Croton On Hudson, NY (US)

(72) Inventor: Robert SZPICZYNSKI, Croton On Hudson, NY (US)

(21) Appl. No.: 16/684,850

(22) Filed: Nov. 15, 2019

(51) Int. Cl. A63B 24/00; G16H 20/30

(52) U.S. Cl. CPC A63B 24/0062; A63B 24/0075; A63B 2225/20; A63B 2024/0068; G16H 20/30

(57) ABSTRACT

Systems and methods configured to allow for planning, recording, storing, reviewing, and analyzing of physical activity of users. The system includes at least one server and a plurality of user terminals. At least one special user terminal is associated with specific piece(s) of exercise equipment and at least one general user terminal is associated with a plurality of pieces of exercise equipment. Each user terminal is configured to allow users to quickly, easily, and intuitively record workout data, and the system is configured to analyze the data so as to provide relevant information to users.

### FIELD OF INVENTION

The present invention generally relates to exercise recording devices and related methods, and in particular to multi-user device(s) configured to allow for planning, recording, storing, reviewing, and analyzing physical activity of users and related methods.

### BACKGROUND OF THE INVENTION

Tracking/monitoring of physical activity is conventionally provided by wearable fitness trackers/monitors and software applications for smartphones/computers, which communicate with each other by, for example, Bluetooth®. A typical fitness tracker/monitor collects physical activity metrics, such as distance walked, calories burned, and heartbeat. Since the screen of a typical fitness tracker/monitor is relatively small and provides the user with limited opportunity for reviewing fitness data, such device transmits the data to a smartphone or computer. The smartphone/computer's fitness application allows a user to store the collected data and review the user's progress/history. Many of these applications allow sport coaches to create and push custom-specific workout schedules directly to their client's fitness applications. Many fitness applications also include “a virtual coach” that can provide suggestions for a workout routine.

Although the fitness tracker/monitor—smartphone/computer system may be adequate for dedicated users engaged in sport activity/exercise, it has drawbacks. For example, some people don't feel comfortable bringing their smartphones to a sport facility. Instead, such people choose to bring sketchpads with a description of their workout schedules and make notes of their workouts there. Also, there are certain types of sport facilities where smartphones are restricted due to privacy/security considerations, as well as certain scenarios where professional athletes undergo regular training sessions and a coach wishes to use one uniform tracking system for all athletes.

Thus, there is an established need for a system that can allow a user to easily log their workouts without the need for conventional fitness tracking monitors and associated software applications. Such a system may advantageously include convenient access to a training schedule, the ability to record physical activity, such as specific exercises, track calories burned, and access workout histories. Such a system can also satisfy the need for a system for monitoring sport activity that securely protects user data.

### SUMMARY OF THE INVENTION

Various illustrative embodiments of the present disclosure provide a system and method for optimizing and tracking user exercise. According to one aspect of the present disclosure, the system comprises: a server; at least one special user terminal (SUT) communicating with the server, the SUT having a user interface and a recognition module; and at least one general user terminal (GUT) communicating with the server, the GUT having a user interface and a recognition module. Each of the user terminals (UT) communicates with the server wirelessly, e.g., via Wi-Fi or Bluetooth® radio frequency, or via wired connection. The system may further include at least one identification device, per user, usable by a user of the system, that is configured to interact with the user terminals.

For each user, the special user terminal is configured for recording information of a specific designated piece of exercise equipment, for example, a weight machine. Alternatively, the special user terminal may be configured for recording information of up to four pieces of exercise equipment. Since the special user terminal relates to a single piece (or up to four pieces) of exercise equipment, it allows a user to quickly and easily enter workout related data of that specific piece of exercise equipment without the need to select a particular piece of exercise equipment from among a list of options.

For each user, the general user terminal allows for recording information of a plurality of pieces of exercise equipment, for example, all exercise equipment located in a room of a gym. The general user terminal may also be configured to record additional information, such a logging which users are accessing the device.

According to embodiments, the recognition module of each user terminal is configured to identify individual users, and comprises, for example, a near-field communication (NFC), radio frequency identification (RFID) device, a QR code reader, a face/voice recognition device, or the like. The identification of an individual user may be provided by a user's corresponding identification device, such as a corresponding NFC and RFID tag, a quick response (QR) code or face/voice recognition. In this way, a user of an exercise facility can quickly and easily log into a specific user terminal by merely connecting their identification device to the user terminal or by voice/face recognition.

In accordance with an aspect of an illustrative embodiment of the present disclosure, the system is configured for use by a plurality of different user types. The user types include, but are not limited to: users of gym equipment, workout coaches or other persons who provide training services to users, staff and/or managers of a workout facility, and third parties, such as organizations, corporations, advertising agencies and manufactures of exercise-related products and services, or the like, that promote products or services.

The system is configured such that users can enter and save a piece of exercise equipment specific data, on a given user terminal, including the number of repetitions and sets, the load (weight), information related to the individual user's pre-existing medical conditions and physiological limitations; retrieve and review recorded equipment specific data; access a workout schedule for a current or future session; modify a workout schedule for a current or future session; access date specific and equipment specific user information, such as calories burned while using the specific piece of equipment; access a workout schedule generated by the system based on analysis of a user's progress and workout history; and access data from other devices, such as an electronic device that has communicated with or is other compatible with the system.

According to embodiments, reviewed information can be configured to be represented on to a user numerically, graphically, or combinations thereof. Such representations can be provided to a user on a separate electronic device, such as a tablet, smart phone, or computer, or alternatively on a user terminal. Additionally, any of the above information may be automatically accessible by another user, such as a personal coach.

According to aspects of the invention, the system is configured to provide users, such as personal coaches, with the ability to transmit a workout schedule to an account of a specific user; access a workout schedule of users and modify said schedule; monitor the progress of users; keep a register of users; monitor areas of interest of users; and access user medical data, such as pre-existing conditions and physiological limitations.

According to embodiments, the system allows users, such as staff and/or managers of a workout facility, to collect data about time and frequency of use of different pieces of exercise equipment from the user terminals. This information may be used to help management optimize equipment location, estimate use and repair needs of equipment, determine what equipment to remove, replace, or duplicate, and more generally understand how their equipment is used. This information also helps management proactively forecast and plan exercise equipment associated capital expenditure. Similarly, such information enables third parties that are interested in promoting industry-related products and services among individual clients and personal coaches, so as to deliver targeted information about their products and services directly to the potential consumers.

According to embodiments, individual users of the system can restrict access of their workout information, such that it is only viewable to a select number of users. The restriction of access can be accomplished via a user's account.

According to embodiments, the system is configured to receive and process data from weight-, motion-, acceleration-, and/or position- (e.g., range of movement) sensors installed on exercise equipment, such as a weight machine. In other embodiments, the system may include infra- or conventional video cameras with image recognition, which are configured to visually record user exercise. Such video cameras are well suited for monitoring of exercises implementing, for example, dumbbells, kettlebells and barbells where other types of conventional sensors are difficult to use.

At least one advantage of the present systems and methods relates to the collection and analysis of reliable data based on actual histories of user workouts. For that reason, in accordance with aspects of the invention, the present systems and method are configured to select, verify and process information from a plurality of users in such a way that workout schedules for each unique user (i.e., users of different sex, age, body physique, pre-existing medical conditions and other pertinent parameters) can be automatically generated.

According to the other aspect of the disclosure, the system is configured to be compatible with wearable fitness trackers/monitors, such as the Apple Watch® and Fitbit®, which record physiological information, such as heartbeat rate, in real time and process physiological information in order to improve the efficiency of user-specific workout schedules.

The system is further configured to provide advertisements to the user terminals while they are not in use, which are displayed for users as they pass by.

According to a further aspect of the disclosure, methods of using the system include a user logging into a user terminal, such as interacting with a recognition module, and upon a successful match the user is successfully identified. The user terminal provides on its touchscreen display graphical images of exercises, associated with a particular piece of exercise equipment that the user terminal is configured for use with. The user then selects a desired exercise and may be allowed to review a history of past exercises of the user (e.g., individual client) for the particular piece of exercise equipment on the touchscreen display. After the individual client uses the particular piece of exercise equipment, he/she enters and saves the number of sets and repetitions and the weights for the current session. The data is then saved to the system and the user's account. Subsequently, the system generates a graphical and/or audio operation completion confirmation.

### BRIEF DESCRIPTION OF THE DRAWINGS

FIG. 1 is a block-diagram of system for recording exercise activities, according to embodiments of the disclosure.

FIG. 2 is a block-diagram of a user terminal of the system, according to embodiments of the disclosure.

FIG. 3 is a block-diagram of a user identification module of a user terminal, according to embodiments of the disclosure.

FIG. 4 is a diagram illustrating types of users which interact with the system, according to embodiments of the disclosure.

FIG. 5 is a diagram of a method of using a special user terminal of the system, according to embodiments of the disclosure.

FIG. 6 is a diagram of a method of using a general user terminal of the system, according to embodiments of the disclosure.

FIG. 7 is a diagram of a further method of using a general user terminal of the system, according to embodiments of the disclosure.

FIG. 8A-C illustrate the graphical user interface of a special user terminal's (SUT) touchscreen display, according to embodiments of the disclosure.

FIG. 9A-E further illustrate the graphical user interface of general user terminal's (GUT) touchscreen display, according to embodiments of the disclosure.

### DETAILED DESCRIPTION

Detailed embodiments of the present systems and methods are disclosed herein; however, it is to be understood that the disclosed embodiments are merely illustrative of the systems, and methods of use thereof that may be embodied in various forms. In addition, each of the examples given in connection with the various embodiments of the systems and methods are intended to be illustrative, and not restrictive. Further, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the systems and the associated methods.

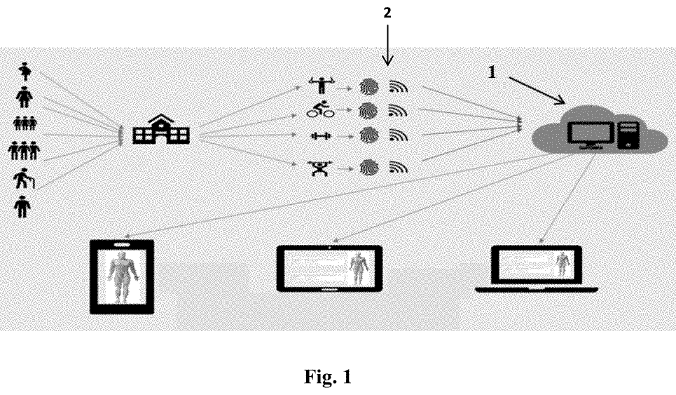

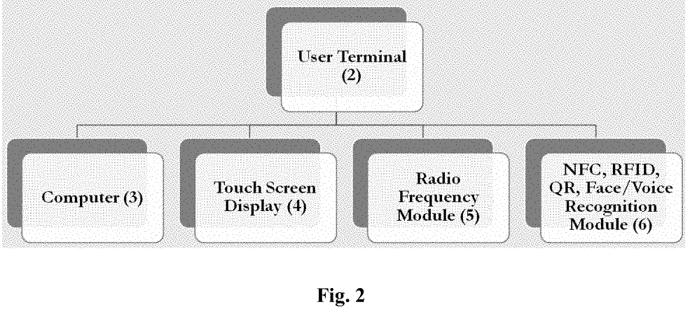

With reference to FIGS. 1 and 2, the system comprises a server 1, a plurality of user terminals 2, and a plurality of electronic devices (e.g., a smart phone, tablet, and/or personal computer). According to embodiments, the server 1 is a computer configured to receive and transmit data, as well as perform computational analysis. In another embodiment, the server 1 is connected to a public or private cloud configured to provide data analysis for the electronic devices and user terminals 2.

According to illustrative preferred embodiments, the system includes user terminals 2, as illustrated by FIG. 2. The user terminals 2 are configured to communicate with the server 1 wirelessly, such as via RF communication, or via a wired connection. The user terminal 2 comprises a computer 3; a display 4; a radio frequency module 5; and a recognition module 6.

The recognition module can take the form of an NFC-, RFID-, QR-, voice recognition, face recognition, or fingerprint recognition device configured to allow a user to log into the terminal. Such a log in occurs when a user provides their own unique code (e.g., NFC, RF, or QR code), speaks to the user terminal, has their face scanned or their finger print scanned. User terminals 2 comprise two types of terminals, a special user terminal and a general user terminal.

The special user terminal 2 is configured to be associated with one or up to four pieces of exercise equipment, for example, a stand alone weight machine, and allows users to enter their workout-related data and review workout histories specific to said piece of exercise equipment. The general user terminal may be associated with a plurality of pieces of exercise equipment, associated exercise equipment of an entire room, or associated with exercise equipment of an entire facility (e.g., gym). The general user terminal similarly allows users to enter their workout-related data and review workout histories from a plurality of pieces of exercise equipment.

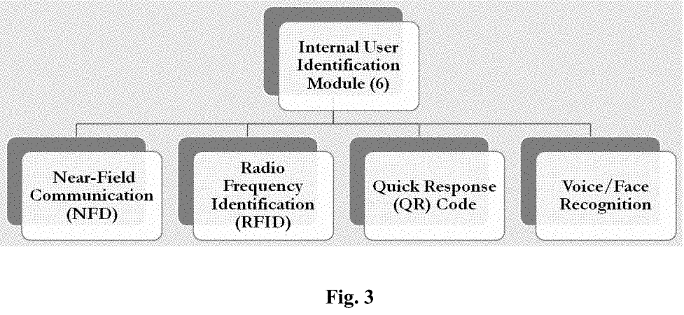

According to embodiments, and with reference to FIG. 3, users of the system set up an account prior to entering workout-related data. For example, users can create a user name and password such that they have a unique account for the system. Additionally, each user's account is associated with unique identification information, such as a unique RFID or NFC tag, a unique QR code, or voice, facial, or fingerprint information. Each user's account can be used to log workout-related data and other user information, and access to such information may be limited to the owner of the account. Users may permit their account information to be accessed by other, as further described below. Once an account is created, a user may “log in” to a user terminal. Logging in can be accomplished, for example, by a user placing a personal near-field communication (NFC) device or radio frequency identification (RFID) device near the user terminal 2 such that the recognition module 6 records the identification information stored on the user's NFC or RFID device. According to one alternative embodiment, the user can scan a unique QR code on the user terminal 2. According to a still further embodiment, the user can speak to or otherwise scan their face or fingertip on recognition module 6 in order to log into the user terminal. By way of example only, the NFC devices may be implemented as a water proof rubber wrist band or a key card to provide visitors with a convenient and secure means for accessing lockers, opening doors, and making in-facility payments. Such an NFC device, according to embodiments of the present disclosure, can be programmed to provide a user of the present system with access to user terminals 2.

According to embodiments, as further discussed below, user terminals 2 may be configured to allow users to manually enter workout data. The workout data can be entered via a graphical user interface provided on display 4.

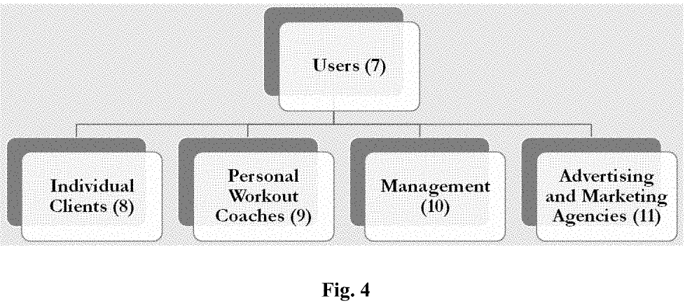

With reference to FIG. 4, the system is configured to be uniquely used by an array of different users 7. The system is configured to allow individual clients 8, e.g., members of a workout facility, to keep detailed records of their workout sessions, and to receive the services of a built-in “virtual coach” via user terminals 2 (or electronic device compatible with the system). The system is configured to allow an individual client 8 to enter and save workout-related data, such as a piece of exercise equipment that the individual client 8 exercises on (with corresponding date and time), the magnitude of a load/weight (lb., kg.) used on the exercise equipment, and the number of sets and repetitions. During the creation of an individual client account within the system, the individual client 8 may input relevant medical conditions, perceived physiological limitations, and other relevant personal information in order to minimize the risk of health-related problems. The system also provides an individual client 8 with an opportunity to access a workout schedule generated by a “virtual coach”, or by a conventional personal coach for each past, current or future available session; and modify an individual workout schedule for a current as well as for any available future session. Notifications of the modifications may be automatically provided to an individual client and/or their coach.

According to alternative embodiments, user terminals 2 may be configured to automatically receive data, for example, via radio frequency, from sensors, such as weight-, motion-, acceleration-, and/or position- (aka. range of movement) sensors, that have been incorporated or are otherwise integrated with exercise equipment. The user terminals 2 are thus configured to automatically log a user's workout on a specific piece of exercise equipment without having the user manually enter the data. Such integration simplifies the process of entering and saving the data as well as makes the data more accurate.

In certain embodiments, the user terminals 2 are equipped with a camera (e.g., a regular- or infrared spectra camera) with image recognition. The camera is configured to record user exercise (e.g., as an alternative to the use of integrated sensors) and log exercise related data to the system. Such embodiments are particularly well suited for automated monitoring of exercises where integrated sensors are ill suited, such as exercise with dumbbells, kettlebells and barbells. The recorded video may be used by the system to identify user range of motion, automatically calculate the number of repetitions and sets, estimate calories burned, and enter such data into the user's account. Certain information may be entered into the user terminals 2, such as the weight of the dumbbells, kettlebells, barbells, etc.

In certain embodiments, the system is configured to calculate the amount of calories burned by the client in each exercise and automatically save such information to the individual clients account.

The system is also configured to allow remote access, such that an individual client and/or a personal coach may access a user account remotely via any electronic device compatible with the system, such as, for example, a smartphone, iPad®, laptop, or a computer.

In some embodiments, a virtual coach is configured to generate a workout schedule for an individual client based on workout related data, and by analyzing the individual client's progress. Such data may also be used to periodically provide adjustments to future workout schedules. According to an individual client's preferences, account information, such as workout related data, can be presented on a user terminal 2 (or personal electronic device) numerically, graphically, or by the combination thereof.

It is to be noted that reference to individual clients 8 is not mean to be limited to regular visitors of a sport facility, but rather incorporate potential individual clients, i.e., people visiting a sport facility to assess its quality and making a decision regarding enrollment. In this way, the system is configured to help assess a current (or prospective) member of a gym person's current fitness level, and provide information on ways to improve physical health and workout routines, such as information about neglected muscles, possible workout schedule(s) and expected progress. Such information can be used to entice new membership by prospective clients.

With reference to FIG. 4, and according to certain embodiments, the system is configured such that it allows a personal coach 9 to push a workout schedule to an account of an individual client. To develop a fully client-specific, efficient workout schedule the personal coach may be authorized by the system to access an individual client's account information, such as physiological and health-related data. According to embodiments, authorization is given by the individual client, for example, via an authorization procedure. The system is further configured to generate a docket (e.g., a list of clients and corresponding workout regimes/schedules with related time and date information) for personal coaches to keep track of individual clients. Once a workout schedule is pushed (e.g., downloaded onto the individual client's account by the personal coach), the system may be configured to periodically remind the coach to review the client's progress and/or adjust it. In one embodiment, the personal coaches accounts may be viewable by individual clients, so as the individual clients may inspect the accounts of personal coaches. Such a feature provides individual clients with the ability to review personal coach workout plans, as well as review personal coach qualification information.

With further reference to FIG. 4, the system provides management of a sport facility 10 with statistical data on the time and frequency of use of exercise equipment within the facility, thereby allowing the management to run the facility more efficiently and effectively. For example, by evaluating time and frequency of use of exercise equipment, estimates can be made regarding future maintenance of equipment, unused equipment can be identified and remove/replaced, popular equipment can be identified and duplicated, etc. According to certain embodiments, the system is configured to generate and analyze the statistical data on the time and frequency of use of exercise equipment. For example, the system can log each time a specific piece of equipment is used, and generate numerical and/or graphical information conveying the statistical data. Additionally, based upon manufacturer information and historical failure rates, the system can analyze the statistical data in order to determine and provide an estimated time of failure for the pieces of equipment.

The system is further configured to enable organizations promoting industry-related manufactured goods and services, including marketing and advertising agencies 11, to both receive relevant market data and directly reach out to individual clients and personal coaches, such as by providing direct advertising on the user interfaces 2. For example, marketing and advertising agencies 11 may have their own accounts, which allow them to receive certain client and coach information. Such information may include, time and frequency of use of exercise equipment and client demographic information (e.g., age, sex, etc.).

The system is further configured to enable an administrator to access the system. Such an administrator may maintain the system, provide software updates, and the like.

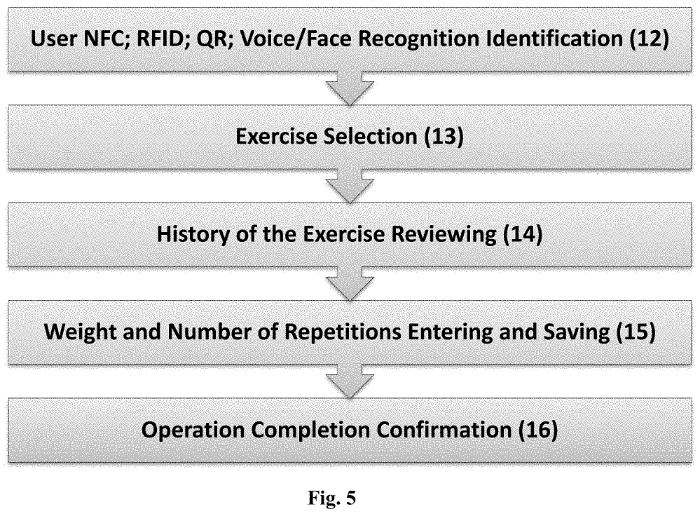

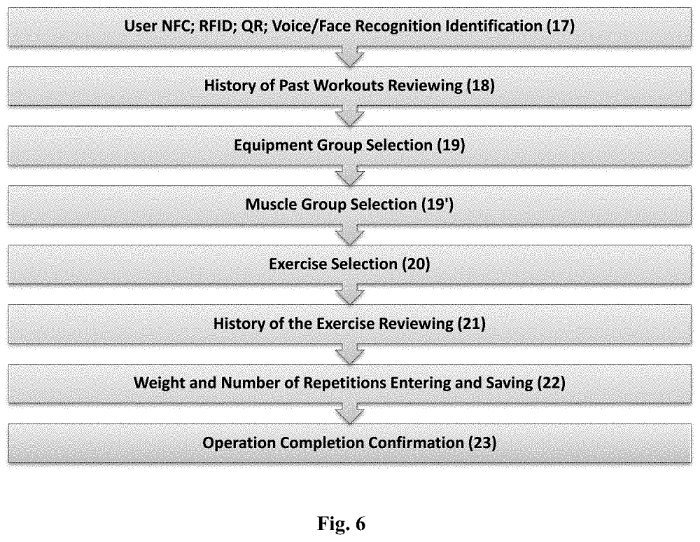

Further aspects of the present disclosure relate to methods of using the system. FIG. 5 illustrates embodiments of a user, such as an individual client 8, interacting with a special user terminal 2. A first step 12 involves the individual client logging into (i.e., being identified) the special user terminal 2. This is accomplished by the user interacting with user recognition module 6 such that the user is identified by the system. According to embodiments, an individual client applies his/her individual NFC- or RFID- or QR key to the special user terminal. According to alternative embodiments, recognition module 6 includes voice/face/fingertip recognition functions, and identification of the user is effectuated by the user speaking, via facial recognition, or via a fingertip scan. The client's unique identification information (i.e., key or speech pattern/facial features) are compared to a database of users to determine which user is attempting to log in. Upon a successful match the user identification is acknowledged by the system and the user is then successfully identified. According to preferred embodiments, the system is configured to compare the client's unique identification information to the database of users after the user selects a desired exercise and enters workout related information (i.e., steps 13-15). In this way, the users unique identification information and workout related information are simultaneously transmitted to the system (e.g., to server 1), and only upon a successful match is the workout related information saved to the user's account. By simultaneously transmitting the unique identification information and workout related information after the user has exercised, the user advantageously does not have to wait for the system to confirm the user's account. The special user terminal provides on its touchscreen display graphical images of exercises, associated with a particular piece of exercise equipment, that the specific user terminal 2 is configured for use with. A second step 13 involves the individual client selecting a desired exercise by touching the pertinent graphical image on the touchscreen display. A further optional step 14 includes presenting a history of past exercises of the individual client for the particular piece of exercise equipment on the touchscreen display. Although the individual client can navigate his/her entire personal history, in a default mode, if the individual client did this particular exercise in the past, the date, the number of sets and repetitions, and the weights pertinent to this last exercise may be provided on the display. After the individual client uses the particular piece of exercise equipment, he/she enters and saves the number of sets and repetitions and the weights for the current session pertinent to the earlier selected piece of exercise equipment at step 15. After the data are successfully entered and saved, the system generates a graphical and/or audio operation completion confirmation 16. The amount of calories burned during the session may also be automatically calculated and added to the user's account. It is to be noted that the specific steps of FIG. 5 are illustrative of a method of using the special user terminal, and certain of the steps do not need to be performed in the aforementioned order. For example, the user can perform their exercise before logging into the terminal. However, the steps of logging in, making a selecting of exercise equipment, and entering and saving workout data are to be performed within a set period of time. In this way, if a user logs into a terminal and does not save workout data within the set period of time, the terminal is configured to log the user out and revert back to a home screen, such that a new user can log in and use the terminal.

With reference to FIG. 6, methods of using the general user terminal by an individual client begins with the individual client identification 17. This step can be the same or equivalent to step 12 discussed above with regard to interacting with a special user terminal 2. Once the identity of the individual client is verified, and in step 18, the general user terminal delivers to its touchscreen display graphical images of exercises earlier performed by the individual client. If the individual client does not want to review her/his previous visits, she/he selects a previously done exercise, or chooses a new equipment group, according to step 19, by touching the corresponding graphical image on the display. After a new equipment group is selected, the individual chooses a muscle group he/she wants to exercise, according to step 19′. Finally according to step 20, the individual client chooses a specific exercise and then exercises with the selected piece of equipment. Thereafter, a history of past exercises can/may be delivered to the touchscreen display, according to step 21. In a default mode, if the individual client did this exercise before, the data pertinent to this last exercise is shown on the display. Upon completion of the exercise, she/he enters and saves the number of sets and repetitions, and the weights for the current exercise 22. Once the data are successfully saved, the system provides the individual client with a graphical and/or audio confirmation 23. The individual client can instruct the system to calculate and keep the record of the calories burned during the session. It is to be noted that the specific steps of FIG. 6 are illustrative of a method of using the general user terminal, and certain of the steps do not need to be performed in the aforementioned order. For example, the user can perform their exercise before logging into the terminal. However, the steps of logging in, making a selecting of exercise equipment, and entering and saving workout data are to be performed within a set period of time. In this way, if a user logs into a terminal and does not save workout data within the set period of time, the terminal is configured to log the user out and revert back to a home screen, such that a new user can log in and use the terminal.

According to alternative embodiments, a step of manually entering workout related data (e.g., steps 15, 22) is omitted. In these alternative embodiments, sensors are associated or otherwise integrated with the exercise equipment and configured to generate the workout related data automatically. The data is then transmitted to the user terminal and saved to the user's account. According to at least one variation thereof, instead of sensors, at least one camera is implemented to record images/video of the user exercising with the piece of equipment and is configured to automatically generate the workout related data and transmit it to the user terminal.

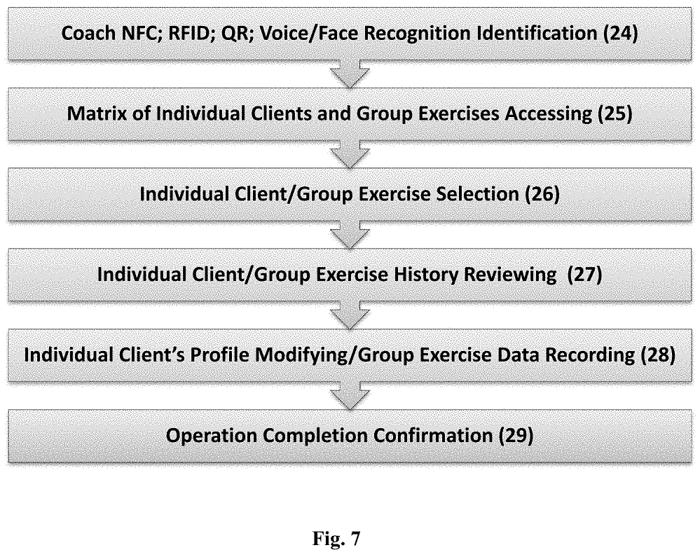

With reference to FIG. 7, methods of using the general user terminal by a personal coach begins with the coach identification 24. This step can be the same or equivalent to step 12 discussed above with regard to interacting with a special user terminal 2. Once the identity of the coach is verified, and in step 25, a matrix of group exercises is provided on the touchscreen display. Accordingly, the personal coach may select any group exercise the coach wants to run for a group of individual clients, according to step 26. According to certain embodiments, the group of clients are those that have enrolled in the class/group by entering their unique user identification information. After selecting the desired element of the matrix, the personal coach may review the exercise histories of individual clients to whom he/she provides the services, or history of group exercises he/she ran in the past, according to step 27. By having access to the account of an individual client, the coach may selectively push a workout schedule to his/her client(s). Additionally, the coach can modify an existing schedule for a regular individual client as well as review client exercise history. A further step 28 can be used to push a workout schedule to a group of clients, for example if the coach runs classes or group exercises, such that participating individual clients automatically receive pertinent exercise-related data to their accounts. The system further generates an operation completion confirmation 29 on a display when the coach has completed data entry and review. An optional step includes each individual client 8 enrolling in a class/exercise group. Such enrollment can be carried out, for example, by each individual client 8 logging into a user terminal 2 and selecting a class.

The coach may also meet a group of his clients, and picks this group from a list on his electronic device. He can then choose an exercise schedule for this specific group, check the attendance and confirm the weigh and reps for each exercise performed by the group. After the group finishes its exercises the coach can then send the schedule of performed exercises to the each individual account of each participant.

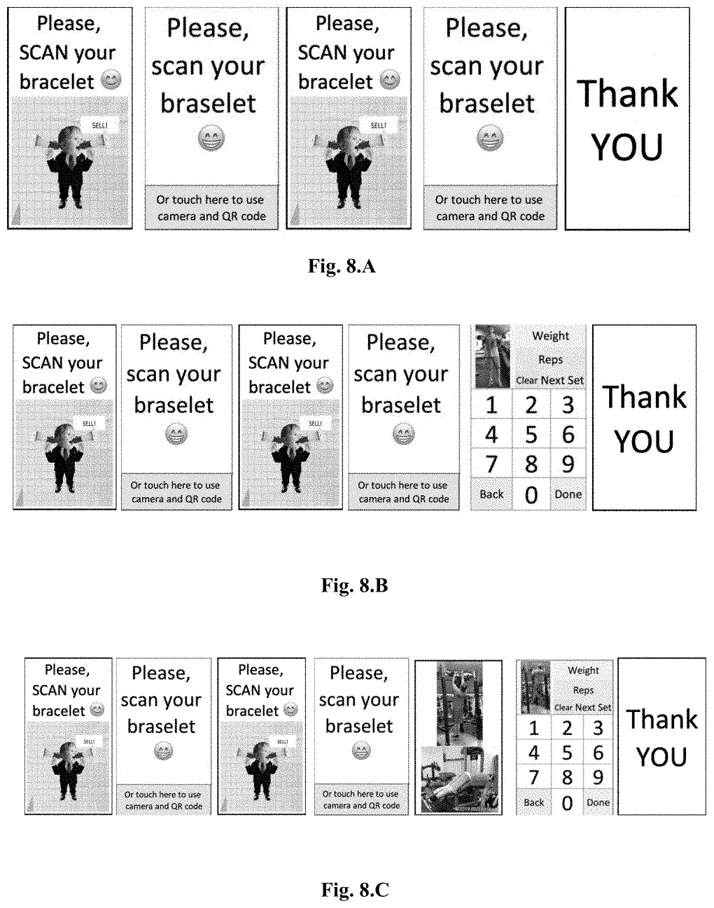

With reference to FIG. 8A-8C, screenshots of the graphical user interface (“GUI”) from a special user terminal's touchscreen display are illustrated, according to illustrative embodiments of the invention. FIG. 8A depicts images appearing on the touchscreen display of a SUT designated to a preset class (e.g. spinning) prompting a user to log in or otherwise be registered with the user terminal. As illustrated, the user is prompted to apply his/her individual key (or use speech/facial recognition) to the special user terminal (e.g., scan a RFID enabled ID bracelet). Once the key (or speech pattern/facial analysis) is identified, all the information set on the SUT about the class (amount of time spinning, distance, speed etc.) is sent to the user's account. The GUI may subsequently provide the user with a confirmation, such as graphically presenting “Thank you”, providing an audio confirmation, like a beep, or the like.

FIG. 8B depicts images appearing on the touchscreen display of a SUT designated for a singular exercise. An individual client logs in to the user terminal, then enters data about her/his exercise, such as weight, number of sets and repetitions, pertinent to the piece of exercise equipment that the user has registered with via the special user terminal. FIG. 8C illustrates a modification to that as shown in FIG. 8B, in that the piece of exercise equipment is configured for more than one distinct exercise. When such a piece of equipment is used, the special user terminal 2 provides an additional display illustrating the different exercises that can be carried out by the exercise equipment, and allows the user to select the exercise that they will perform (or already performed) prior to entering exercise data, such as weight, number of sets and repetitions.

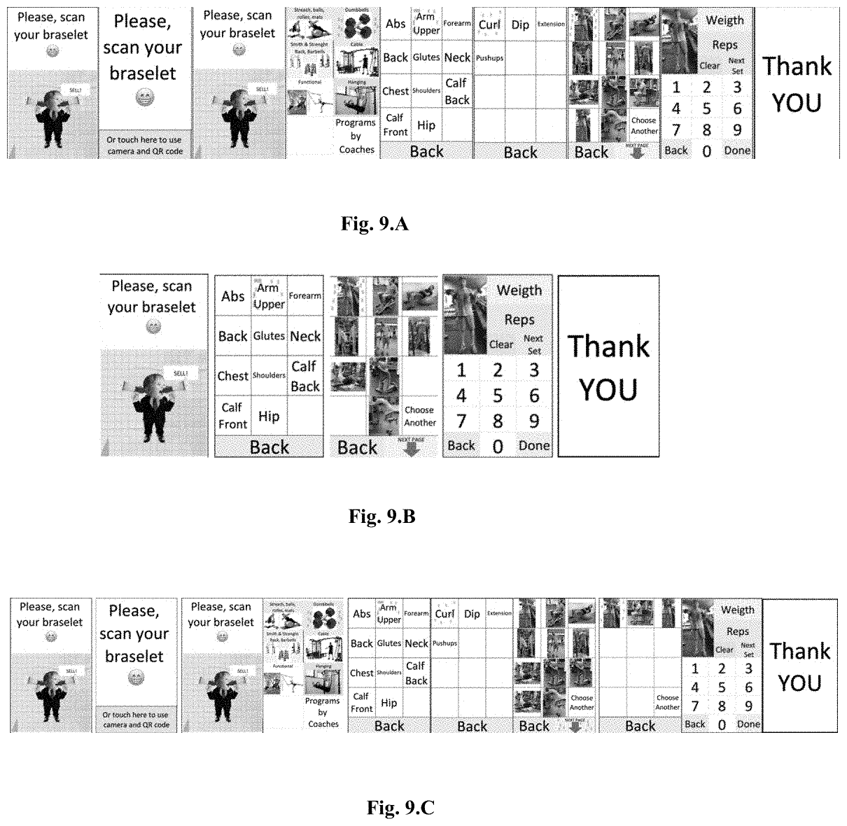

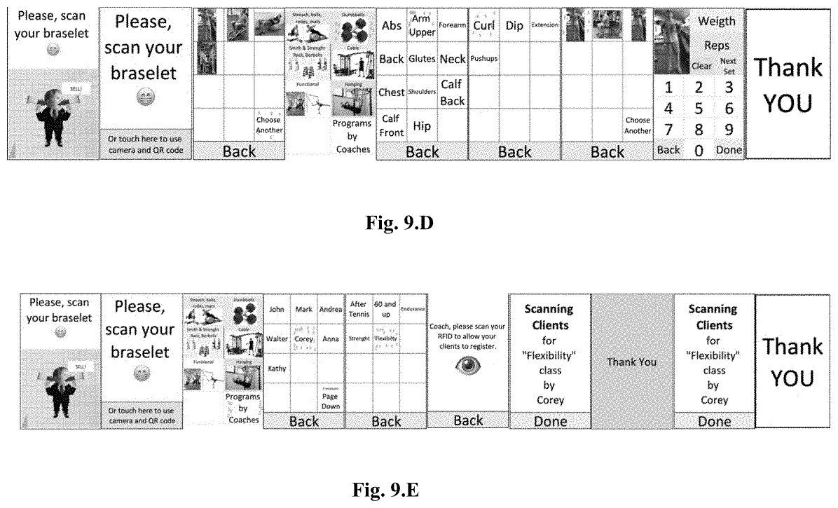

With reference to FIG. 9A-9E, screenshots of the GUI from the general user terminal's touchscreen display are illustrated, according to illustrative embodiments of the invention. FIG. 9A depicts images appearing on the touchscreen display of a general user terminal 2 configured for use with an array of exercise equipment. As FIG. 9A illustrates, once the user has registered with the user terminal they are prompted to select a particular equipment group, then muscle group then particular exercise and then enters data pertinent to the exercise, such as weight and number of sets and repetitions. Once the exercise information is entered a visual confirmation, such as “Thank you” may be displayed. FIG. 9B, depicts images appearing on the touchscreen display of a general user terminal providing a historical list of exercises, such as exercises performed on a previous visit. FIG. 9C illustrates how a user can scroll through a list of exercises. For example, when the list of exercises associated with a general user terminal is large, multiple pages are implemented, such that a user scrolls through multiple pages, e.g., by pressing “Next Page” button. FIG. 9D is an extension of FIGS. 9B and 9C. In this scenario, the user may want to choose another exercise not include in the history. In this case the user chooses “Choose another” and continue with a regular selection of the exercise. Upon the completion of the exercise, the client enters and saves exercise data.

FIG. 9E depicts images appearing on the touchscreen display of a general user terminal configured for use and is an extension of FIG. 9D. If an individual client presses the button “Programs by Coaches”, she/he is provided with a matrix of individual coaches available and group exercises. After that the individual client selects (e.g., enrolls) in a desired group exercise that will take place in the future by applying her/his key to the terminal. In some embodiments, the group exercise related images on the display are accompanied by numbers showing the vacancies available for the enrolment (not shown on FIG. 9). Additionally, the general user terminal may be configured such that a client cannot enroll in a class without authorization from a coach. As FIG. 9D also illustrates, a coach may have to provide authorization, for example, via scanning of an RFID tag, in order for the client to enroll.

Examples of how the system and related methods are implemented will be described below. However, it is noted that these examples are merely provided to be illustrative and are in no way meant to limit the disclosure.

Example 1: An individual client enters the room for group training (e.g. a spinning class), and registers by applying their individual key to the user terminal. When the session is over, either the designated individual coach enters into the system the time and duration of the session or the SUT is preprogrammed. The exercise data is then automatically uploaded to the personal accounts of the participating individual clients.

Example 2: An individual client is performing an exercise of 3 sets, 10 repetitions each with 50 lb. load, on a weight machine that provides several distinct exercise options. Upon completion of all three sets, the client accesses the system by applying his/her individual key to the special user terminal 2. From several available options he/she then selects the pertinent exercise and enters the following data: weight=50; sets=3(or inputs three separate sets via selecting “next set”); rep.=10 on the touchscreen display, and saves them. This exercise data is now stored in the client's account and is accessible for review.

Example 3: An individual client is performing an exercise of 2 sets, 10 repetitions each with 50 lb. load, on a weight machine that provides several distinct exercise options. Upon completion of all two sets, the client accesses the system by applying his/her individual key to the special user terminal 2. From several available options he/she then selects the pertinent exercise and enters the following data: weight=50; rep.=10 on the touchscreen display, selects “next set”, inputs and weight=50; rep.=10, and then saves them. This exercise data is now stored in the client's account and is accessible for review.

Example 4: An individual client is doing an exercise of 3 sets, 10 repetitions each with 50 lb. load, on a weight machine that provides a single exercise only. Upon completion, the client accesses the system by applying her/his individual key to the terminal and enters the following data: weight=50; sets=3 (or inputs three separate sets via selecting “next set”); rep.=10 on the touchscreen, and then saves it. The saved data is now stored in the client's account and is accessible for his/her review at any time.

Example 5: After finishing exercising by carrying out arm curls of 3 sets, 5 repetitions each, with a load of 25 lb. exercise, an individual client accesses the system by applying the client's individual key to a general user terminal. The system identifies all exercises which the client did in this area of the premise in the past, and if the client already did the upper arm curls earlier, this exercise would be listed in the client's account. The client then selects the upper arm curl exercise and enters the following data: weight=25; set=3 (or inputs three separate sets via selecting “next set”); rep=5 on the touchscreen and saves it. If this is the first time client did an upper arm curl, then this exercise is not already listed in the client's account, and the client first have to find the needed exercise in the system's docket, selects the exercise, and then centers and saves the information about the exercise, including number of sets, repetitions and the weight. Although, the system may contain several hundred exercises, they can be categorized my muscle, exercise type, and the like, providing user-friendly searching.

1. A system for inputting and tracking user exercise, comprising:
a server;
at least one special user terminal configured to communicate with the server, comprising:
a first user interface; and
a first recognition module,
wherein the at least one special user terminal is configured to record exercise activity for at least one piece of exercise equipment,
at least one general user terminal configured to communicate with the server, comprising:
a second user interface; and
a second recognition module,
wherein the at least one general user terminal is configured to record exercise activity for a plurality of pieces of exercise equipment, the plurality of pieces of exercise equipment being greater in number than the at least one piece of exercise equipment.

2. The system for inputting and tracking user exercise of claim 1,
wherein each of the at least one special user terminal and the at least one general user terminal further comprise a recognition module configured to identify a user.

3. The system for inputting and tracking user exercise of claim 2,
wherein the recognition module comprises:
a near-field communication device;
a radio frequency identification device;
a QR code reader;
a facial recognition device;
a speech recognition device;
a fingerprint recognition device; or
combinations there.

4. The system for inputting and tracking user exercise of claim 1,
wherein each of the at least one special user terminal and the at least one general user terminal is configured to record equipment specific exercise data, comprising: type of exercise;
number of repetitions; number of sets; exercise weight; or combinations thereof.

5. The system for inputting and tracking user exercise of claim 4,
wherein the specific exercise data is configured to be transmitted to and displayed on an electronic device, the di splay being numerical, graphical, or combinations thereof.

6. The system for inputting and tracking user exercise of claim 1,
wherein the system is configured to create an account for each user, and
wherein the system is configured to provide at least one user with a workout schedule, and modify at least one workout schedule.

7. The system for inputting and tracking user exercise of claim 4,
wherein the system is configured to determine, utilizing the equipment specific exercise data, time and frequency of use data of the at least one piece of exercise equipment, the plurality of pieces of exercise equipment, or both.

8. The system for inputting and tracking user exercise of claim 7,
wherein the system is configured, utilizing the time and frequency of use data:
to estimate a time of failure of the at least one piece of exercise equipment, the plurality of pieces of exercise equipment, or both, and
to determine which exercise equipment to remove, replace, duplicate, or combinations thereof.

8. The system for inputting and tracking user exercise of claim 1, further comprising:
at least one sensor configured to record exercise data, the exercise data comprising: load data, motion data, acceleration data, position data, or combinations thereof.

9. The system for inputting and tracking user exercise of claim 4, further comprising:
at least one video capturing device configured to record images of user exercise,
wherein the recorded images are utilized to determine the equipment specific exercise data.

10. A method of tracking user exercise, comprising:
creating an account for each of a plurality of users, wherein the account, for each user, is associated with unique user identification information;
allowing a user to log into a user account by providing the unique user identification information to a special user terminal or a general user terminal, wherein the at least one special user terminal is configured to record exercise activity for at least one piece of exercise equipment, and wherein the at least one general user terminal is configured to record exercise activity for a plurality of pieces of exercise equipment, the plurality of pieces of exercise equipment being greater in number than the at least one piece of exercise equipment.
providing, on the special user terminal or a general user terminal, a graphical user interface, the graphical user interface allowing the user to record equipment specific exercise data, comprising: type of exercise; number of repetitions; number of sets; exercise weight, or combinations thereof; and
confirming and saving the equipment specific exercise data to the respective user's account.

11. The method of tracking user exercise of claim 10,
wherein the unique user identification information comprises:
a numerical key;
a QR code;
a user speech pattern;
a facial pattern;
a fingerprint; or
combinations thereof.

12. The method of tracking user exercise of claim 11,
wherein special user terminal and general user terminal comprises a recognition module comprising:
a near-field communication device;
a radio frequency identification device;
a QR code reader;
a facial recognition device;
a speech recognition device;
a fingerprint recognition device; or
combinations there,
wherein the unique user identification information is provided to the recognition module.

13. The method of tracking user exercise of claim 10, further comprising:
determine, utilizing the equipment specific exercise data, time and frequency of use data of the at least one piece of exercise equipment, the plurality of pieces of exercise equipment, or both.

14. The method of tracking user exercise of claim 13, further comprising:
estimating, utilizing the equipment specific exercise data, a time of failure of the at least one piece of exercise equipment, the plurality of pieces of exercise equipment, or both; and
determining, utilizing the equipment specific exercise data, which exercise equipment to remove, replace, duplicate, or combinations thereof.

15. The method of tracking user exercise of claim 10, further comprising:
recording exercise data, the exercise data comprising: load data, motion data, acceleration data, position data, or combinations thereof, from at least one sensor.

16. The method of tracking user exercise of claim 10, further comprising:
recording image data of user exercise,
wherein the recorded images are utilized to determine the equipment specific exercise data.

17. The method of tracking user exercise of claim 10, further comprising:
providing on the graphical user interface of the special user terminal or a general user terminal a history of the recorded equipment specific exercise data.

18. The method of tracking user exercise of claim 10, further comprising:
providing at least one advertisement on the graphical user interface.
